(19) United States
(12) Patent Application Publication
(10) Pub. No.: US 2009/0171363 A1
(43) Pub. Date: Jul. 2, 2009

(54) AUTOMATED INSERTION DEVICE FOR HEART VALVE PROSTHESIS

(75) Inventor: Sidney CHOCRON, Besancon (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: UNIVERSITE DE FRANCHE-COMTE, Besancon (FR)

(21) Appl. No.: 12/341,292

(22) Filed: Dec. 22, 2008

Related U.S. Application Data
(60) Provisional application No. 61/080,001, filed on Jul. 11, 2008.

(30) Foreign Application Priority Data
Dec. 28, 2007 (FR) 0760444
Feb. 29, 2008 (FR) 0851346
Jun. 20, 2008 (FR) 0854081

(51) Int. Cl. A61B 17/58; A61B 17/00; A61F 2/24; A61B 17/10

(52) U.S. Cl. 606/99; 606/206; 606/142; 623/2.1

(57) ABSTRACT

The invention concerns an automated ancillary device (100) for inserting and fixing by means of clips an annular body (200), namely a prosthesis, on a annular resilient volume (23) including, coaxial about an axis (D), means for receiving and supporting (8) said body (200) coaxial to its axis, means for radially unfolding (500) about said axis (D) articulated supports (4) capable of bearing clips (21), the latter being capable of passing through both a thickness of material belonging to such a volume (23) and a thickness of material belonging to said body (200), under the action of means for longitudinally pushing (600) said supports (4) with respect to said means for receiving and supporting (8).

It includes means for gripping (300) material of the volume (23) capable of radially retracting said material. The means for radially unfolding (500) are capable of moving said clips (21) parallel to said axis (D).

### BACKGROUND OF THE INVENTION

(1) Field of the Invention

The invention relates to an automated ancillary device for inserting and fixing a prosthetic cardiac valve.

The present invention relates to the field of the surgery tools.

The invention relates more particularly to an automated ancillary device for inserting and fixing a prosthetic cardiac valve. A prosthetic cardiac valve is a prosthesis with a circular or annular shape, which is implanted instead of a patient's heart valve, in particular an aortic valve or a mitral valve.

The surgical operation during which the practitioner resects, i.e. cuts and removes the patient's native valve or valves so that only the patient's ring remains, i.e. an annular surface belonging to the heart to which the prosthetic valve will be fixed, then implants and fixes a prosthetic cardiac valve, is long and delicate. Indeed, in order to carry out a replacement of a natural valve by a prosthetic valve it is necessary to bring about an extra-corporal circulation of the patient's blood. It is interesting to reduce to a minimum the duration of this extra-corporal circulation, which is not good for the patient. Indeed, an extended duration can cause particular clinical complications.

The invention thus proposes to put at the disposal of the heart-surgery practitioners an ancillary device, which permits them to considerably reduce the duration of the operation, so as to reduce the risk of complications.

(2) Description of the Prior Art

In a known way, a prosthetic cardiac valve is fixed by stitching. One proceeds to stitching the prosthetic valve by means of separated stitches between the prosthetic valve and the ring.

The surgeon thus often proceeds to stitching at least about twenty separated stitches in order to fix the prosthetic cardiac valve. In order to carry out these stitches, he performs the fixing of the valve with respect to the ring that remains at the level of the patient's heart, in the way of a parachute, i.e. he draws rather long double wires between the patient's ring and the prosthesis. Once he has passed the wires through all these stitching points, for example, about twenty stitching points, he draws all the wires together, which permits him to bring the prosthetic valve close to the patient's ring, he can thus apply the prosthesis against the ring. He then fixes the wires by making a knot and he cuts all the wires. This operation is long, takes usually 45 to 60 minutes and is delicate.

Two usual stitching methods are known:
inserting at simple separate points: through the ring are passed at regular intervals needles fitted on wires in the ventricle-aorta or ventricle-auricle direction, depending on the cardiac valve considered. On the other end of the wire is also fitted a needle, which is passed through the collar, generally of “Dacron”, of the prosthesis. Once all the wires have been passed through the patient's ring and the collar of the prosthesis, the prosthesis is lowered, in the way of a parachute, into contact with the ring and the wires are tied.
inserting at separated points in the form of a U on pledgets: the wire is with two needles and a 3 mm long and 1 mm wide supporting felt. The two needles are passed in the ventricle-aorta or ventricle-auricle direction, depending on the cardiac valve considered, then passed through the collar of the prosthesis, the prosthesis is lowered, in the way of a parachute, into contact with the ring and the wires are tied.

These operations are time-consuming for a type of surgery in which time is important. Indeed, while carrying out these gestures, the heart is stopped, and blood circulation as well as its oxygenation depend on the extra-corporal circulation (ECC).

Various attempts to accelerate the operational time have been made. An alternative to the traditional device for stitching a prosthetic valve on the patient's ring is a stent incorporating the prosthetic valve. The stent is itself closed in the form of a cylinder having a diameter of about 8 mm, this stent is inserted through the femoral artery and is moved upwards to the heart. The disadvantage is that this stent is necessarily fixed in the patient's native valve, and that a proper cutting of the patient's valve cannot be performed. The native valve is generally very hard because of the accumulation of calcium, and its walls are porous and are no longer elastic. Placing the stent in the patient's native valve is a bad solution for this tightness problem. This solution is reserved for patients who cannot be operated. In addition, the prostheses inserted into such stents are new, and there is no knowledge as to their longevity. On the other hand, the ancillary device according to the invention permits to very reliably use the prostheses that have been existing for a long time and the life time of which has been proven.

The invention relates, as a matter of fact, to providing means permitting the practitioner to operate so-called non operable patients, often more than 75 years old, and who could become operable if the operational time is highly reduced with respect to the state of the art.

Further attempts have been made while improving the stitching method. U.S. Pat. No. 6,413,274 B1 thus describes a device for placing, at the level of the patient's ring, a crown of clips implanted in radial directions with respect to the axis of the patient's ring to which are connected the ends of the wires inserted in the prosthetic valve. This method allows a reduction of the operational time, the practitioner must however still carry out all the finishing of the stitching work. In addition, the clips are implanted at the periphery of the patient's ring, and a risk of tear of the tissues cannot be excluded. A document U.S. Pat. No. 6,464,707 describes a similar system.

US2001/0031972 A1, in turn, describes an apparatus for placing a prosthetic valve through fastening with clips, which is designed so as to unfold the clips radially with respect to the axis of the patient's ring. These clips include, each, two tapping ends. The first one is straight and is designed capable of perforating a collar the prosthetic valve includes, after radial extension of a clip holder. The other tapping end is bent and is designed capable of separating the tissues during the radial extension of the clip holder, then of perforating same during its deformation towards a closing position enclosing the tissues in the vicinity of said collar, under the action of the translation of a tube inserted into the tissues. It is obvious that the use of such a device damages the tissues during the insertion of the tube in order to close the bent end, and that the straight end is inserted at a very short distance from the surface of the patient's ring. Thus, in these two areas there exists a risk of tear, which should absolutely be avoided. This solution does not guarantee a complete closing of the clip around the prosthesis and the patient's ring, at the level of the tissues there is a fragile area in the vicinity of the two points of the clip when the latter is bent. The problem is that, as a matter of fact, this zone in which the clip is not fully closed is also the most strained by the blood pressure. In short, the method using the device according to this document US2001/0031972 A1 does not permit to transfix the patient's ring, but only to perform a simple hooking, with a high risk of damaging the tissues.

Another document US2006/217744 describes foldable arms to implant a stent in an artery or the like.

Another document WO 2005/048883 describes an apparatus for placing a prosthetic valve through fastening with clips. These clips have a first tapping end in the prosthetic valve, and a second one in the patient's ring. These second one is anchored like a partially opened hook in the patient's annulus, and there is, too, a risk of tear of the tissues.

Another documents US2006/212114, US2001/044656, WO97/30659 describe means and methods of replacing a heart valve in a minimally invasive manner.

### SUMMARY OF THE INVENTION

The purpose of the invention is to cope with the disadvantages of the state of the art, and to very substantially reduce the operational time by providing the surgeon with an ancillary device that allows him to perform, in a fully reliable way for the patient and without any risk of tear for the tissues, the placing of a prosthetic cardiac valve within about 10 to 15 minutes, thanks to the use of an automated ancillary device for inserting and fixing a prosthetic cardiac valve.

The invention pretends to allow the automation of the insertion and the placing of the prosthetic cardiac valve, in order to make them easier and to allow the practitioner to save time. The aim looked for is also to facilitate a robotized implanting, or first through a small path, where these operations become long and painful.

Of course, the development of a prosthetic cardiac valve is extremely delicate and extremely long. Thus, the aim is not to substitute new models for the prosthetic cardiac valves existing on the market, but to use the valves which have proved reliable and which are hitherto accepted by almost all patients.

To this end, the invention relates to an automated ancillary device for inserting and fixing by means of clips an annular body, namely a prosthetic cardiac valve, on a substantially annular resilient volume, said ancillary device including, coaxially fitted about a longitudinal axis, means for receiving and supporting said annular body designed capable of receiving the latter coaxially to its axis of rotation, means for radially unfolding about said longitudinal axis articulated clip supports designed capable of bearing clips that are designed capable of passing through both a thickness of material belonging to such an annular volume and a thickness of material belonging to said annular body under the action of longitudinal pushing means of said clip supports with respect to said means for receiving and supporting, wherein it also includes means for gripping material of said annular volume designed capable of radially retracting said material, and wherein said means for radially unfolding about said longitudinal axis said articulated clip supports are designed capable of moving said clips parallel to said longitudinal axis.

According to a feature of the invention, said means for radially unfolding about said longitudinal axis said articulated clip supports are designed capable of moving said clips parallel to said longitudinal axis, and include to this end an aggregate of pantographs each formed of two rods each articulated, at one of their ends, to one of said clip supports and, at their other end, respectively to a nut guided in an outer tube and indexed in rotation on the latter, on the one hand, and to an ferrule, on the other hand, which nut and ferrule are both internally threaded and designed capable of cooperating with threads in opposite direction, which a screw with an axis designed capable of being driven by a rotary ring with an axis includes.

According to a feature of the invention, said gripping means are mounted coaxially to said longitudinal axis.

The major advantage of the invention is to permit, through the implementation of the ancillary device, a complete closing of each clip on itself, by enclosing in a durable way the various elements fastened together by the clip, after having exerted a pulling force on one of the elements fastened together by the clip in order to secure the latter against any successive tear. In the preferred case of fixing a prosthesis on a resilient annular volume, such as a cardiac ring, the ancillary device according to the invention permits to ensure that each clip passes completely through the prosthesis and also through this annular volume.

Further features and advantages of the invention will become clear from the following detailed description of the non-restrictive embodiments of the invention, with reference to the attached figures.

### BRIEF DESCRIPTION OF THE DRAWINGS

FIG. 1 represents, schematically and partially, a cross-sectional view, according to its longitudinal axis, of an ancillary device according to the invention, shown provided with means for receiving and supporting, in the form of a bell, an annular body;

FIG. 2 represents, schematically and in perspective, the means for receiving and supporting an annular body, in the form of a bell, of FIG. 1;

FIG. 3 represents, schematically and from an end, the bell of FIG. 2;

FIG. 4 represents, schematically, partially and in cross-section, a first position of a ancillary device according to the invention, including means for gripping inactivated material;

FIG. 5 is a view similar to the preceding one, representing a second position of the ancillary device of FIG. 4, in which these gripping means are activated under the action of a depression;

FIG. 6 is a view similar to the preceding one, representing a third position of the ancillary device of FIG. 4, in which means for radially positioning the clips are separated from the longitudinal axis;

FIG. 7 is a view similar to the preceding one, representing a fourth position of the ancillary device of FIG. 4, in which clip supports are brought close to a resilient annular volume under the action of pushing means, for inserting clips into the latter;

FIG. 8 is a view similar to the preceding one, representing a fifth position of the ancillary device of FIG. 4, in which said clip supports are brought close to an annular body for making it integral, by means of the clips, with the annular volume;

FIG. 9 represents, schematically and in perspective, a clip made out of <<Nitinol>> at a first temperature;

FIG. 10 represents, schematically and in perspective, the clip of FIG. 9 at a second temperature;

FIG. 11 represents, schematically and in longitudinal cross-section, the means for gripping material of figures A to E;

FIG. 12 represents, schematically, an another clip shown at different temperatures.

### DETAILED DESCRIPTION OF THE PREFERRED EMBODIMENTS

The invention relates to an automated ancillary device 100 for inserting and fixing an annular body 200, namely, in a preferred application, a prosthetic cardiac valve, on a substantially annular resilient volume 23 and including an opening. In fact, in a natural state, this resilient volume, when it is formed by the patient's ring, generally has a triangular cross-section with rounded angles. This volume 23, after a preparation by the surgeon to remove very hard areas, is shaped and a cylindrical tool, called phantom, is passed through it, which permits to define the diameter of the prosthesis, the latter being generally circular.

A preferred embodiment of the invention is shown in the figures.

The annular body 200 includes at least one revolving portion rotating about an axis of rotation. It is preferably formed of a prosthetic valve, or a biological valve. Such prostheses or valves are as follows: having a diameter from 19 to 33 mm, they permit, as regards the smallest ones, the insertion through the center of an ancillary device having a diameter preferably smaller or equal to 13 mm, which at the end of the procedure can be withdrawn in the same way. The biological prostheses account for 60% of the currently implanted prosthetic valves. The ancillary device 100 according to the invention is designed for the placing of a type of proven prosthesis, characterized by a high rate of absence of re-intervention, of about 90% over a 15 years' period.

The ancillary device 100 according to the invention permits a method of placing of the annular body 200 no longer by stitching as in the prior art, but by fastening with clips 21, which is perfectly safe in use and can be performed with a high accuracy. The automated ancillary device 100 constitutes a device for fastening an annular body 200, namely a prosthetic valve, in particular a cardiac valve, on a substantially annular resilient volume 23, namely a patient's ring prepared by a surgeon.

The ancillary 100 comprises a shaft 24. In the axis of this shaft 24, a device 25, retractable in the way of an umbrella, preceded by an ogival insertion ferrule 1, is designed capable of being inserted, in closed position, into the substantially annular volume 23.

This retractable device 25 is designed capable of being unfolded, namely inside the patient's ventricle, below a partition formed by the substantially annular volume 23, namely a patient's ring, with respect to the upper side from which it is inserted.

The diameter size, with respect to a longitudinal axis D the ancillary device 100 includes, of the retractable device 25 is preferably smaller or equal to 13 mm. It is recalled that the diameter sizes of the usual prostheses are range from 25 to 33 mm for mitral valves and from 19 to 27 mm for aortic valves.

The ancillary device 100 includes, designed capable of being mounted coaxially about a longitudinal axis D:
means for receiving and supporting 8 an annular body 200, which are designed capable of receiving the latter coaxially to its axis of rotation, and of bringing it applied against the body 23,
means for radially unfolding 500, around this longitudinal axis D, articulated clip supports 4. The latter are designed capable of bearing clips 21. These clips 21 are designed capable of passing through several thicknesses, i.e. both a wall of a substantially annular volume 23, namely formed by tissues, and the annular body 200, namely the prosthesis, under the action of longitudinal pushing means 600. These longitudinal pushing means 600 result, under the action of a force exerted at the level of a control handle 16 provided with a gripper 17, into bringing the clip supports 4 closer to said means for receiving and supporting 8, or also to the control handle of the ancillary device 100.

The ancillary device 100 is thus designed to bind together a substantially annular volume 23, namely formed the patient's ring, and an annular body 200, by means of a network of clips 21 designed capable of being closed around this substantially annular volume 23 and this annular body 200, under the action of a force exerted on one or several clip supports 4, on the one hand, or/and on these means for receiving and supporting 8, or under the action of a force exerted by the clips 21 themselves, during a change of physical state.

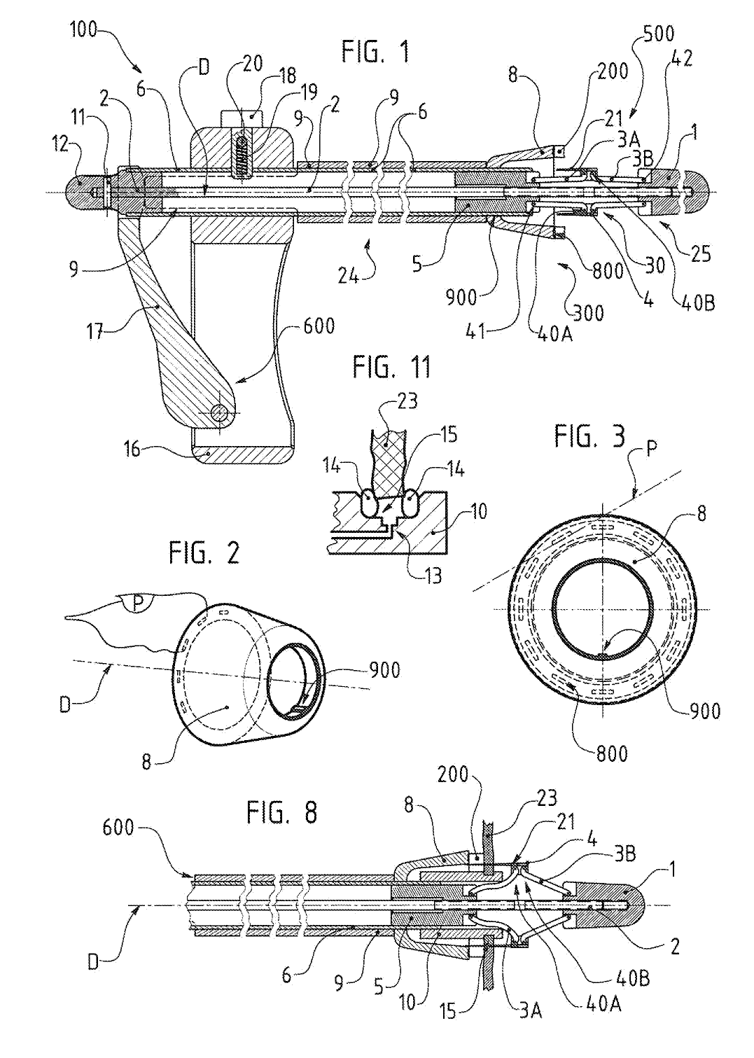

These means for receiving and supporting 8, as can be seen in FIG. 1 or 2, are preferably designed with a shape and size capable of receiving an annular body 200 formed by a prosthetic cardiac valve. They are namely formed, in a version shown in FIG. 1, by a bell 8 capable of acting as an anvil, and which, in a particular version, includes means for guiding the clips for their closing 800, as can be seen in FIG. 3.

In a preferred way, the network of clips 21 is annular, and rotating about the longitudinal axis D. It can also be star-shaped, centered about this same axis.

The quality of the fastening by clips is directly related to the proper positioning of the clips 21 supported by the clip supports 4, with respect to the substantially annular volume 23. Indeed, if the diameter on which the clips 21 are placed is too close to the opening of this substantially annular volume 23, there exists a risk of tearing the wall of the latter. This substantially annular volume 23 is resilient, i.e. has some elasticity. According to the invention, the latter is used, when designing the ancillary device 100 capable of exerting a radial pulling force on the substantially annular volume 23, namely the patient's ring, before performing the fastening by means of clips.

To this end, the ancillary device 100 according to the invention includes means for gripping 300 material, namely at the level of a wall, pertaining to the substantially annular volume 23, which are designed capable of performing a radial retraction, towards its axis, of the material of the volume 23 in the vicinity of its opening.

Preferably, these gripping means 300 are mounted coaxially to the longitudinal axis D.

As can be seen in FIG. 1, the gripping means 300 are formed, in a preferred embodiment, by a ring 10, and include suction means designed capable of exerting a radial or/and axial depression at the level of the material of the volume 23 in the vicinity of its opening.

These suction means preferably consist of one or several grooves or orifices 13, provided for at the bottom of a rotation recess 15 designed capable of receiving the volume 23 in its full thickness, each orifice 13 being connected, through at least one channel, to a suction group so sized as to exert in said orifice or orifices 13 a depression with a radial or/and axial component. The radial depression is preferred, since it permits the radial retraction of the volume 23 until its free end rests in the recess 15. Preferably, as can be seen in FIG. 11, this recess 15 includes one or several resilient seals 14, designed so as to ensure the tightness while the volume 23 is maintained under depression at the bottom of the recess 15. In a particular variant embodiment, the ring 10 itself can be made out of resilient material and ensure this tightness around the volume 23. In a preferred application, the depression is calculated so as to permit a radial retraction of the radius of the volume 23 by a few millimeters, preferably by 2 to 3 millimeters. The width of the recess 15 is, preferably, larger than 4 millimeters.

It is obvious that the orifices 13 can be made in the form of grooves, because of the simplicity of their carrying out, they can also consist of points of depression, or the like, namely radially arranged in a star arrangement.

This possibility of retraction of the substantially annular volume 23, which the ancillary device 100 according to the invention provides, is particularly important in the preferred application for heart surgery. It permits to prevent the patient's ring from being pierced by clips over a small thickness close to its opening, thus exposing it to tearing when it is subjected to the forces of the blood flow.

The clips 21 must then be accurately positioned with respect to the substantially annular volume 23, at the points of fastening by clips determined by the user.

To this end, the means for radially unfolding 500 articulated clip supports 4 about the longitudinal axis D are arranged at the level of the retractable device 25. These means 500 include an aggregate of pantographs 30 comprised, each, of two rods 3A, 3B, each articulated at one of their ends to one of the clip supports 4 and, at their other end, respectively to a nut 5 guided in an outer tube 6 and indexed in rotation on the latter, on the one hand, and to a ferrule 1, on the other hand, the nut 5 and the ferrule 1 being both internally threaded and designed capable of cooperating with threads in the opposite direction, which a screw 2 with an axis D designed capable of being driven by a rotating ring 12 with an axis D includes.

The articulated clip supports 4 are preferably each articulated to rods 3A, 3B forming together an articulated pantograph 30 extending radially according to a plane passing through the longitudinal axis D.

Preferably, as can be seen in FIG. 1, the shaft 24 of the ancillary device 100 includes an outer tube 6 with a longitudinal axis D. This tube 6 constitutes means for guiding, at a first end, a tapped nut 5. This nut 5 is designed capable of circulating in the tube 6, and first rotation-indexing means oblige this nut 5 to circulate only longitudinally according to the direction D. Preferably, these first rotation-indexing means are formed by the conjugation of a longitudinal slit parallel to D, which the outer tube 6 includes, which slit is designed capable of cooperating with a radial pin, which the nut 5 includes.

The nut 5 is provided with radial recesses and hinging means 41, namely pins, for permitting the free hinging of first rods 3A, which are in turn, each, fixed by means of pins with a view to the free hinging, at the level of hinging means 40A, of a clip support 4. This nut 5 cooperates, at the level of its internal thread, with a first thread of a threaded spindle 2, designed capable of being driven in rotation, at the end opposite this first end, by a rotary ring 12, namely a knurled ring, through a pin 11, the rotary ring 12 being guided by the outer tube 6 on which it is mounted so as to rest on it.

The threaded spindle 2 also includes a second threaded portion the pitch of which is in a direction opposite that of the first threaded portion, and which is designed capable of cooperating with an inner thread, which the insertion ferrule 1 includes, which ferrule is provided, like the nut 5, with recesses for articulated rods 3B at the level of hinging means 42, namely pins, rods 3B, which are each hinged, at the level of hinging means 40B, to a clip support 4. The hinging means 40A and 40B are distant from each other. Preferably, the lengths between the hinging 41 and 40A, on the one hand, 42 and 40B, on the other hand, are identical, and the axes of the hinging means 40A and 40B are equally distant from the axis D. It is thus obvious that any rotational motion imparted by the rotary ring 12 to the threaded spindle 2 results into translations according to the direction D, but in opposite direction, of the nut 5 and the ferrule 1. Indeed, the latter are connected in a hinging way by the rods, can have no rotational motion about D, neither with respect to each other, nor with respect to the outer tube 6. The rods 3A and 3B can be unfolded or folded, and drive the clip support 4 according to a radial movement, always parallel to the axis D, as can be seen in FIG. 6. In addition, because of this configuration, the radial movement always occurs in the same plane perpendicular to the axis D.

The action of the means for radially unfolding 500, about the longitudinal axis D, through unfolding the articulated pantographs 30 thus permits to move the clip supports 4, and thus the clips 21 they support, exclusively parallel to the longitudinal axis D, in a preferred embodiment. The means for radially unfolding 500 also permits to accurately position the clips 21 on a selected annular diameter. Of course, the means for controlling these means for radially unfolding 500, in particular at the level of a knurled rotary ring 12, can advantageously be graduated.

Thus, by providing the clip supports 4 with equally long clips 21, it is possible to control the accurate radial position of the tapping end the latter include. The clips 21 are preferably double clips, i.e. including, connected to a heel designed capable of cooperating with a clip support 4, two parallel tapping legs perpendicular to this heel. They are preferably made out of titanium alloy. In an advantageous variant, the clips 21 are made out of shape-memory <<Nitinol>> alloy, so as to spontaneously close after passing through the substantially annular volume 23 and the annular body 200. They are then capable of being closed under the action of a shape-memory trigger, which can be thermal, electric, or mechanical. In the latter case, the bell 8 is no longer absolutely necessary in the ancillary device during the closing of the clips 21, and is necessary only for bringing the annular body 200 into its position of fastening by clips in the vicinity of the volume 23.

It is obvious that the proper holding of the clips 21 is essential, is particular when the ancillary device 100 is used in heart surgery. The ancillary device 100 permits to position the clips 21 absolutely parallel to the axis of the substantially annular volume 23, according to which the axis D of the ancillary device is aligned. For a perfect holding at the moment of the closing of the clips 21, it has been devised to make special clips with a large heel, i.e. with a height of about 2 mm, for clips with a total length of 9 mm, in a preferred embodiment.

The clips 21 arranged in the form of an annular beam can thus be brought very accurately into the insertion position in front of the substantially annular volume 23, and can then be inserted through the latter, according to a relative movement with a direction D of the tube 6 with respect to the substantially annular volume 23, thanks to the previous action of the gripping means 300, which guarantees a sufficient material cross-section in order to prevent any tear between each clip 21 and the radial edge of the substantially annular volume 23 towards the axis D.

The ancillary device 100 comprises, designed capable of being mounted coaxially about the axis D, means for receiving and supporting 8 the annular body 200, which are designed capable of receiving the latter coaxially to its axis of rotation. These means for receiving and supporting are, preferably and as can be seen in FIG. 1, formed by a rigid bell 8 coaxial to the axis D, and designed capable of being driven longitudinally by a pushing tube 9 with an axis D, at a first end of this tube 9.

This bell 8 includes, in a particular application in which the clips 21 are folded by percussion during plastic deformation, second means for indexing the rotation 900 with respect to the outer tube 6 on which it is designed capable of sliding, namely under the action on the pushing tube 9. These means for indexing the rotation can preferably be formed by the combination of a pawl, which the bell 8 includes, as can be seen in FIG. 3, and a longitudinal groove, which the outer tube 6 includes. It is thus indexed in rotation, not only with respect to the tube 6, but also with respect to the nut 5, at the clip supports 4 and at the clips 21 themselves.

The ancillary device 100 includes, in a version implementing plastically deformable clips 21, means for longitudinally pushing 600 the clip supports 4 with respect to these means for receiving and supporting 8. The pushing means 600 include a gripper 17 articulated on a movable handle 16 designed capable of being mounted in an integral and indexed way with respect to the outer tube 6, this gripper 17 being designed capable of applying a force according to the direction D to a pushing tube 9 designed capable of transmitting this force to the means for receiving and supporting 8, in opposition to the clip supports 4, which are fixed in position, through the nut 5, with respect to the outer tube 6.

The pushing tube 9 is designed capable of being actuated, at its end opposite this first end, by a gripper 17 articulated at the level of a handle 16. This handle 16 is designed capable of being made integral with the outer tube 6, namely by means of an indexing button 18 provided with springy restoring means 19 and with a holding spindle 20. The pushing tube 9 is also designed capable of cooperating with a recess, namely a perforation, which the outer tube 6 includes.

It is obvious that a force in the direction D, applied to the gripper 17 imparts a relative movement of the pushing tube 9 with respect to the threaded spindle 2 and thus with respect to the clip supports 4. This force is transmitted by the pushing tube 9 to the bell 8 bearing the annular body 200.

The longitudinal pushing means 600 are designed capable of cooperating with guiding means 800 for closing the clips 21, in the version of closing of the clips 21 through plastic deformation. These guiding means 800 are incorporated in the means for receiving and supporting 8. The latter are angularly indexed with respect to the clip supports 4 by indexing means 900.

The bell 8 thus includes, on the side facing the clip supports 4 and facing each of them over a given diameter, so-called diameter of fastening by clips, means for guiding 800 the clips 21 for their closing through plastic deformation under the action of the impact of these longitudinal pushing means 600. These guiding means 800 are formed, at the level of a frontal face of the bell 8 by co-planar left machinings in a plane P parallel to the axis D, facing each other and designed capable of deforming towards each other the two points of each clip 21, these machinings are similar to those existing on an anvil of an office stapler, and are distributed over a frontal annular sector of the bell 8, according to the diameter of fastening by clips. The longitudinal travel distance of the longitudinal pushing means is calculated larger than the distance separating the point of each clip 21 from the bell 8, so as to force each clip point to bend, under the pushing force, while resting on the face nearest to the components fastened together by clips, here the annular body 200. It is obvious that, for means for guiding the clips 800 as can be seen in FIG. 3, each clip 21 folds onto itself in the plane P defined by the corresponding guiding means 800.

Thus, the aggregate of clips 21 is inserted at once by an impact, which brings the bell 8 closer to the clip supports 4. The clips 21 pass completely and successively through the substantially annular volume 23 and the annular body 200, their points then bend on the guiding means 800 and are folded closely against the face of the annular body 200 away from the substantially annular volume 23, after each clip 21 has completely passed through the latter. The heel, which each clip includes, maintained in its clip support 4, remains resting against the substantially annular volume 23.

It is obvious that each bell 8 is designed for a particular diameter of fastening by clips, depending on the radial position of the means for guiding 800 the clips 21 for their closing. The same ancillary device 100 can thus be used for several applications, it is enough to change the bell 8 and to choose the one adapted to the required diameter. This is all the more easy since, as can be seen in the figures, the ancillary device 100 is designed with coaxial subsets inserted onto each other and very easy to be dismantled, cleaned and maintained.

Indeed, as can be seen in FIG. 1, the ancillary device includes, aligned with respect to the axis D, the threaded spindle 2, which the nut 5 cooperates with. While moving radially away from the axis D, the nut 5 is guided in the outer tube 6 on which the pushing tube 9 is guided, acting itself as guiding means, at its end located on the side of the clip supports 4, for the ring 10.

The exchange of a bell 8 is thus particularly easy, as well as that of a ring 10, which can also be chosen of a size depending on the application.

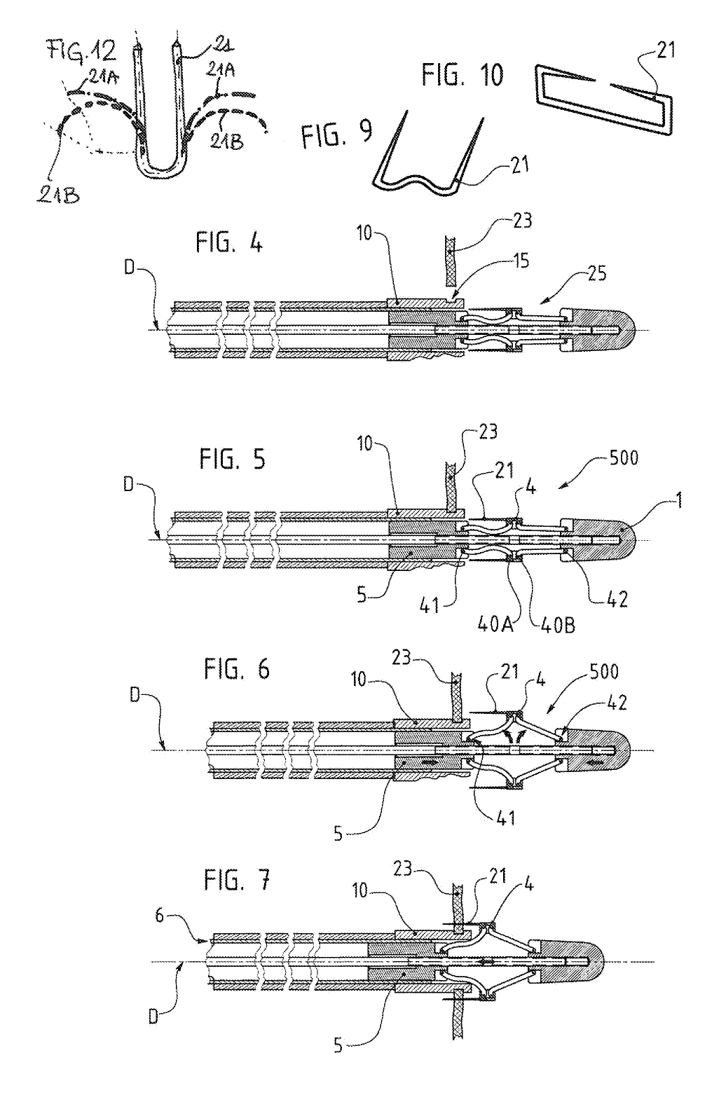

A sequence of clips is shown from FIG. 4 to FIG. 8, it corresponds to the case of the insertion of the ancillary device 100 without the bell 8, of the penetration of the substantially annular volume 23 by the needles 13, as can be seen in FIG. 4, of the retraction of the volume 23 towards the axis D, as can be seen in FIG. 5, and, after unfolding of the clip supports 4, as can be seen in FIG. 6, of the insertion of the clips 21 into the substantially annular volume 23, as can be seen in FIG. 7. The operator then inserts the bell 8 provided with the annular body 200, as can be seen in FIG. 8.

If the operator wants to use plastically deformable clips 21, he installs the removable handle 16 and the gripper 17 at the end of the pushing tube 9. He finally proceeds to impacting the clips 21 onto the bell 8 under the action of the pushing means 600.

If he wants to use shape-memory clips 21, he can at this stage, where the annular body 200 is maintained integral with the volume 23 by the clips 21, close them under the action of a modification permitting their change of state, and namely under the action of a rise in temperature.

Of course, the pushing means 600, here shown in a simplified form, can be provided with effort reducers, namely with an eccentric or knuckle mechanism, in the way of an office stapler.

The clips 21 are preferably deformed over a longitudinal travel distance of about 2 mm. In the case of plastically deformable clips 21, a longitudinal impulse on the outer tube 6 permits, after fastening by clips, to uncouple the heels of the clips 21 from their recesses for the clip supports 4.

In the preferred case of using shape-memory clips, namely made out of <<Nitinol>>, the biocompatibility of which is proven, the bell 8 only serves for holding the annular body 200, and as a support at the base of the clip for the substantially annular volume 23 and the annular body 200, and is not required for closing the clip 21, the closing of which is brought about by a thermal or electric, or mechanical trigger, or the like, which will be inserted so as to cover the bell 8, or instead of the latter.

According to the invention, these clips 21 made out of <<Nitinol>> are designed capable of passing from a first unfolded position at a first temperature to a second folded position at a second temperature higher than the preceding one.

They can, in a reversible movement, pass from the second position to the first position by passing from the second temperature to the first one, thus permitting an easy ablation of the system, should such be necessary.

The shape-memory properties of <<Nitinol>> are then used during the preparation of the clip 21 for its change in state and shape between two temperatures: as can be seen in FIG. 9, at a first low temperature, i.e. in the range of 8° C., the clip 21 has a U-shaped with two substantially parallel legs, which will be inserted into the annular volume 23 and the annular body 200. As can be seen in FIG. 10, at a second temperature, which is higher than the first temperature, for example close to 30° C., applied after complete insertion of the clip 21 into the latter, the legs of the U are bent towards each other, thus enclosing the annular volume 23 and the annular body 200.

The clips 21 advantageously include a heel designed capable of passing from a first folded position at a first temperature to a second unfolded position at a second temperature, which is higher than the preceding one.

In an another execution, this heel is designed capable of passing from a first unfolded position at a first temperature to a second folded position at a second temperature, which is higher than the preceding one.

In every case, the clip 21 and the clips support 4 are designed to allow, during the change of the form of the heel, a good extraction of the clip 21 outside its clip support 4.

In a preferred execution, the heel is designed capable of passing from a first position with a form like a “Z” or like a “S” or incurved at a first temperature, as can be seen in FIG. 9, to a second straight position, as can be seen in FIG. 10, at a second temperature, which is higher than the preceding one.

It is also possible to use these shape-memory properties by providing the heel, through which the clip 1 cooperates with the clip holder 4, at the first temperature a corrugated shape so as to prop up this heel on faces the clip holder 4 includes, and to thus ensure its perfect holding in the latter, as can be seen in FIG. 9, and, at the second temperature, a straight shape permitting its release without friction from the clip holder, as can be seen in FIG. 10.

It is obvious that, thanks to the use of such clips made out of <<Nitinol>>, the bell 8 is not absolutely necessary and can be maintained for acting as a counter-support. Indeed, the shape-memory properties alone already ensure the proper closing of the clip 21 on the annular volume 23 and the annular body 200 together. By maintaining then the body temperature close to 37° C., any reversibility of the shape of the clips 21 is prevented and their permanent fixing is thus guaranteed.

After fastening by means of clips, a reverse operation of the means for radially positioning 500 the clip supports 4 without their contents then permits to fold the latter along the threaded spindle 2, while flattening the pantographs 30.

It is then possible to extract the ancillary device 100 through the opening of the substantially annular volume 23, which is then made integral with the annular body 200.

It is conceivable to carry out the automated ancillary and insertion device 100 for cardiac valve test in two forms, either for disposable use in an embodiment including internally parts made of plastic or in a fully re-usable form, for example made out of titanium alloy or/and of stainless steel or the like, and thus sterilizable.

Such an automated ancillary device 100 can also easily be operated by remote control by a manipulator, since only elementary rotational and translational movements are implemented, at the level of the various control, adjustment and pushing means of this ancillary device. The ancillary device 100 can then also be provided with vision means, namely through optical-fibre endoscopy or the like.

The ancillary device 100 can also be provided with a device for refilling clip 21.

The ancillary device 100 according to the invention can be prone to variations according to results of the experimental surgery, and be used for applications other than heart surgery for which it has been designed. Though the example given here mainly relates to aortic and pulmonary implantations, this ancillary device can apply to implantations on other rings, namely mitral, tricuspid rings, then with modifications due to the direction of clearance of the prosthesis 200. Indeed, in the position of the aortic or pulmonary valve, the clearance of the prosthesis is respectively in the aorta or the pulmonary artery located above the ventricle. In the position of the mitral or triscupid valve, the clearance of the prosthesis is located in the left, respectively right ventricle. These modifications relate to the reversal of the clip supports 4, which will then be located, when referring to FIG. 1, left with respect to the ring 10, the means for receiving and supporting 8, and the annular body 200, and no longer right with respect to same. The ring 10, the means for receiving and supporting 8, and the annular body 200 will then be in the opposite direction, i.e. from right to left, compared to the one shown, i.e. from left to right, in FIG. 1. Likewise, the clip supports 4 will then be oriented in the opposite direction.

Above and on the drawings is described a way where the clips are closed with their first and second end, which go one in direction to another. But, in an other way to use the ancillary device 100 according to the invention, the clips 21 can be used to deform their first and second ends opposite one from another, each in the direction outside of the main member of the clip. This other way allows a better covering of the gaps between the clips, and a good adaptation of the curvature.

In this case, these clips 21, namely made out of <<Nitinol>>, are designed capable of passing from a first folded position at a first temperature to a second unfolded position at a second temperature higher than the preceding one.

A clip 21 usable in this other way is shown in FIG. 12, the clip in wire of “Nitinol” od diameter 0.5 mm has a form like a “U” in low temperature, with a radius of 1.65 mm at the head of the “U”, a distance of 2.5 mm between the two legs of the “U” which are 11 mm long. At a temperature of 35° C., the legs are bent in the position 21A semi-circular outside of the “U” with a radius of 5 mm an an opening angle of 126°. At a higher temperature of 37° C. or more, that is the human body temperature, the final bending in the position 21B has a radius of 4 mm and an opening angle of 157.5°, and is designed to ensure a good support to the prosthesis.

The best mode of the invention is described here. An other way to deform the patient's ring before including the clips, with needles mounted on an expansible ring coaxial with the tube 6, is described in the applications FR 07 60444 and FR 08 51346 of the same inventor.

1. Automated ancillary device for inserting and fixing by means of clips an annular body, namely a prosthetic cardiac valve, on a substantially annular resilient volume including an opening, said ancillary device including, coaxially fitted about a longitudinal axis, means for receiving and supporting (8) said annular body designed capable of receiving the latter coaxially to its axis of rotation, means for radially unfolding about said longitudinal axis articulated clip supports designed capable of bearing clips, which are designed capable of passing through both a thickness of material belonging to such an annular volume and a thickness of material belonging to said annular body under the action of longitudinal pushing means of said clip supports with respect to said means for receiving and supporting, wherein it also includes means for gripping material of said annular volume designed capable of radially retracting said material, and wherein said means for radially unfolding about said longitudinal axis said articulated clip supports are designed capable of moving said clips parallel to said longitudinal axis.

2. Ancillary device according to claim 1, wherein said means for radially unfolding about said longitudinal axis said articulated clip supports are designed capable of moving said clips parallel to said longitudinal axis, and include to this end an aggregate of pantographs each formed of two rods each articulated, at one of their ends, to one of said clip supports and, at their other end, respectively to a nut guided in an outer tube and indexed in rotation on the latter, on the one hand, and to an ferrule, on the other hand, which nut and ferrule are both internally threaded and designed capable of cooperating with threads in opposite direction, which a screw with an axis designed capable of being driven by a rotary ring with an axis includes.

3. Ancillary device according to claim 1, wherein said gripping means are mounted coaxially about said longitudinal axis and are formed a ring with an axis including suction means designed capable of exerting a radial or/and axial depression at the level of the material of said volume in the vicinity of its opening.

4. Ancillary device according to claim 1, wherein said means for radially unfolding about said longitudinal axis said articulated clip supports are designed capable of constituting means for radially positioning said clips.

5. Ancillary device according to claim 1, wherein said longitudinal pushing means include a gripper articulated on a handle designed capable of being mounted integral and indexed with respect to said outer tube, said gripper being designed capable of applying a force according to said direction to a pushing tube designed capable of transmitting said force to said means for receiving and supporting, in opposition to said clip supports fixed in position with respect to said outer tube.

6. Ancillary device according to claim 1, wherein said longitudinal pushing means are designed capable of cooperating with guiding means for closing said clips, which said means for receiving and supporting include, the latter being angularly indexed with respect to said clip supports by indexing means.

7. Ancillary device (100) according to claim 1, wherein said clips are double clips made out of shape-memory alloy and designed capable of passing from a first unfolded position at a first temperature to a second folded position at a second temperature, which is higher than the preceding one.

8. Ancillary device according to claim 7, wherein said clips include a heel designed capable of passing from a first folded position at a first temperature to a second unfolded position at a second temperature, which is higher than the preceding one.

9. Ancillary device according to claim 1, wherein said clips are double clips made out of shape-memory alloy and designed capable of passing from a first folded position at a first temperature to a second unfolded position at a second temperature, which is higher than the preceding one.

10. Ancillary device according to claim 1, wherein said means for receiving and supporting are designed with a form and size capable of receiving an annular body formed by a prosthetic cardiac valve.
